(19) United States
(12) Patent Application Publication
(10) Pub. No.: US 2015/0140570 A1
(43) Pub. Date: May 21, 2015

(54) HIGH-SPEED TWO-STEP INCUBATION METHOD AND APPARATUS FOR IN-VITRO DIAGNOSTIC TESTING

(71) Applicant: ARRYX, INC., Chicago, IL (US)

(72) Inventors: Haojun Fu, Naperville, IL (US); Daniel M. Mueth, Chicago, IL (US); Sergio O. Guevara, Chicago, IL (US); Neil Rosenbaum, Chicago, IL (US)

(21) Appl. No.: 14/384,885

(22) PCT Filed: May 28, 2013
(86) PCT No.: PCT/US13/42848

§ 371 Date: Sep. 12, 2014

Related U.S. Application Data
(60) Provisional application No. 61/652,671, filed on May 29, 2012.

(51) Int. Cl. C12M 1/00; B01L 7/00

(52) U.S. Cl. CPC C12M 41/14; C12M 23/22; B01L 7/00; B01L 7/52; B01L 2300/1805; B01L 2300/0609
USPC 435/7.1; 435/303.1; 435/29; 422/547; 436/501

(57) ABSTRACT

An incubator including a container having an interior chamber capable of receiving a sample holder having samples, a lower heated surface disposed within the interior chamber of the container, and a movement mechanism coupled to the container and the lower heated surface. In one embodiment, the movement mechanism is configured to move the lower heated surface to a position in which the lower heated surface essentially contacts the sample holder. A method of rapidly heating and incubating a sample for biochemical or immunological testing is further disclosed.

### BACKGROUND OF DISCLOSURE

1. Field of Disclosure

Embodiments of the disclosure relate generally to methods and apparatus used to process samples, such as in-vitro diagnostic testing and screening, and more particularly to an incubation method and an incubator to quickly heat samples and maintain the samples at a desired temperature.

2. Discussion of Related Art

Incubators are used to grow and maintain microbiological samples, such as cultures or cell cultures. Most incubators maintain environmental conditions, such as temperature and humidity, to effectively grow and maintain microbiological samples. In its simplest form, an incubator embodies an insulated container or box and an adjustable heater to heat the interior of the container. It is known that biological organisms effectively grow at 37° C. Most incubators have the ability to control the temperature of the container with the adjustable heater.

In in-vitro diagnostic (IVD) testing, incubators are used for process steps which require incubation of a sample or solution for a period of time to enable a reaction to take place. For example, reactions involving antibody-antigen binding occur with different rates and possibly different specificities at different temperatures. For this reason, many diagnostic tests and screening assays for human testing purposes are often performed at 37° C. Thus, elevating a sample from room temperature to 37° C. reliably and accurately, and holding it at 37° C. for a period of time is a common need. This is frequently performed by placing the sample in an incubator, which is held at 37° C., and acts by passive convection or possibly through forced-air convection. This method, while simple and precise, is very slow. For example, even small samples in a wellplate geometry may take 15 to 20 minutes to reach 35° C.

Another method that is commonly used is a water-bath incubator. Water bath incubation is much faster and offers similar or even better thermal uniformity, it is less readily automated and leads to increased costs, complexity, and failure. Further, water bath incubation places distinct constraints on the type of disposable that is suitable.

A third method is to have heat blocks with pockets shaped into them which enables a sample cuvette to fit into the pockets. This method is commonly used for thermal cycling where the sample temperature is repeatedly raised and lowered, such as for PCR. For common IVD testing for non-PCR applications, passive air convection incubators are most common. Forced-air convection incubators are also employed, but offer minimal improvement unless the disposable is suitably designed.

There are applications for which high-speed testing is advantageous. For example, hospital blood banking has a high percentage (approximately 30%) of samples requested as STAT (high priority) test and current testing methods are very slow involving long incubation times. For many assay and disposable formulations, water bath incubation is impractical and thus slow air incubation methods are employed. It is expected that incubation methods which can bring samples to 37° C. more quickly will reduce testing time and improve test performance. Further, high-speed assays place very stringent requirements on incubator performance, since the total incubation time can be as short as several minutes. For this reason, it is critical that high-speed incubation methods be developed. Such technology must be very fast (less than 45 seconds for room-temperature samples to reach 37° C.+/−2° C.), simple, cheap, easily automated, amenable to simple process control and monitoring, needing minimal maintenance, and not significantly changing in performance over time. Further, no portion of the internal lower surface of fluid should ever exceed 37° C.+/−2° C. at any point in time. Despite these numerous constraints, the latest testing instrumentation can offer a degree of flexibility in that custom disposables may be co-developed with the incubator to jointly achieve these requirements.

### SUMMARY OF DISCLOSURE

One aspect of the disclosure is directed to an incubator comprising a container having an interior chamber capable of receiving a sample holder having samples, a lower heated surface disposed within the interior chamber of the container, and a movement mechanism coupled to the container and the lower heated surface. In one embodiment, the movement mechanism is configured to move the lower heated surface to a position in which the lower heated surface essentially contacts the sample holder.

Embodiments of the incubator may include one or more of the following features. In one embodiment, the movement mechanism includes a motor configured to move the lower heated surface between an engaged position in which the lower heated surface engages the sample holder and a disengaged position in which the lower heated surface is spaced from the sample holder. The motor includes an eccentric coupling secured to the lower heated surface. The lower heated surface is positioned below the sample holder. The incubator further comprises an upper heated surface positioned above the sample holder. The sample holder includes a bottom surface configured to mate with the lower heated surface. The interior chamber includes a top chamber having the sample holder, the lower heated surface and the movement mechanism, and a bottom chamber.

Another aspect of the disclosure is directed to a method of rapidly heating and incubating a sample for biochemical or immunological testing. In one embodiment, the method comprises: heating the sample with conductive heat transfer at a first, higher temperature for a short period of time; and incubating the sample at a second, lower temperature for a longer period of time.

Embodiments of the method may include one or more of the following features. In one embodiment, the conductive heat transfer is achieved by bringing a lower heated surface into close proximity or contact to a sample holder configured to hold one or more sample. The lower heated surface and/or the sample holder are designed to achieve essential contact with each other. The heating of the sample with conductive heat is achieved by an incubator comprising a container having an interior chamber, the interior chamber being configured to receive the sample holder having samples, a lower heated surface disposed within the interior chamber of the container, and a movement mechanism coupled to the container and the lower heated surface. The movement mechanism is configured to move the lower heated surface to a position in which the lower heated surface essentially contacts the sample holder. The interior chamber includes a top chamber having the lower heated surface and the movement mechanism, and a bottom chamber. Heating the sample at the first temperature is achieved within one chamber and heating the sample at the second temperature is achieved within the other chamber. The first temperature is greater than 50° C. and less than 90° C. The first temperature is applied for less than one minute. The first temperature is applied for approximately 45 seconds. The second temperature is between 34° C. and 43° C.

Another aspect of the disclosure is directed to an incubator comprising a first chamber held at a first temperature, capable of accepting a sample holder containing a sample, a second chamber, held at a second, higher, temperature, capable of accepting a sample holder containing a sample, and a heated surface, within the second chamber, capable of achieving essential contact with a sample holder containing a sample.

Embodiments of the incubator may include one or more of the following features. The incubator further comprises a mechanism for moving the sample holder between the first chamber and the second chamber. The first chamber is held between 34° C. and 43° C. The second chamber is held between 50° C. and 90° C. A heated surface within the second chamber is within 0.25 mm of a flat lower bottom surface of the sample holder for at least 20 seconds.

Yet another aspect of the present disclosure is directed to an incubation system comprising a transparent sample holder, with a flat lower surface, which is able to achieve essential contact with a flat heating surface without scratching the flat lower surface, and an incubator comprising a first chamber held at a first temperature, capable of accepting the transparent sample holder containing a sample, a second chamber, held at a second, higher, temperature, capable of accepting the transparent sample holder containing a sample, and a heated surface, within the second chamber, capable of achieving essential contact with a sample holder containing a sample.

Embodiments of the method may include holding the first chamber between 34° C. and 43° C., and/or holding the second chamber between 50° C. and 90° C.

### BRIEF DESCRIPTION OF DRAWINGS

Various aspects of at least one embodiment are discussed below with reference to the accompanying figures, which are not intended to be drawn to scale. Where technical features in the figures, detailed description or any claim are followed by references signs, the reference signs have been included for the sole purpose of increasing the intelligibility of the figures, detailed description, and claims. Accordingly, neither the reference signs nor their absence are intended to have any limiting effect on the scope of any claim elements. In the figures, each identical or nearly identical component that is illustrated in various figures is represented by a like numeral. For purposes of clarity, not every component may be labeled in every figure. The figures are provided for the purposes of illustration and explanation and are not intended as a definition of the limits of embodiments of the disclosure. In the figures:

FIGS. 1-5 are schematic cross-sectional views illustrating a method of processing samples;

FIG. 6 is a cross-sectional view of an exemplary lower heating element of an incubator designed to work with wellplates, with the lower heating element employing shaped mating surfaces for the lower heating element to mate with flat-bottom wells with a lower lip at the perimeter of each well;

FIGS. 7a and 7b are cross-sectional views of a disposable well, which has appropriate features to enable rapid incubation in an incubator without a lifting mechanism and optical imaging of the inner lower surface of the well;

FIG. 8 is a perspective view of a portion of an incubator, utilizing a lifting mechanism to contact the shaped mating surface of the lower heating element to bring it in contact with the bottom surface of the wellplate, taken from one side of the incubator;

FIG. 9 is a perspective view of the incubator of FIG. 8 taken from another side of the incubator;

FIG. 10 is a cross-sectional view of the incubator of FIG. 8, taken along line 10-10 of FIG. 8;

FIG. 11 is a perspective view of an embodiment comprising a custom well-strip and incubator jointly designed to achieve high-performance incubation;

FIGS. 12a and 12b are perspective and side elevational views of the well-strip and incubator shown in FIG. 11 with portions removed to illustrate working components of the incubator;

FIG. 13 is a graph showing average temperature of the sample throughout the full sample volume versus time for a sample in a flat-bottom well in the incubator of FIG. 6;

FIG. 14 is a graph showing the temperature-time series for fluid at several points near the lower surface of a flat-bottom well in the incubator of FIG. 6;

FIG. 15 is a view of an exemplary embodiment which utilizes an external transport mechanism to transport the custom well-strip between regions of the incubator; and

FIG. 16 is a view of an exemplary embodiment which utilizes an external transport to move a standard well-plate comprising flat-bottom well-strips between regions of the incubator in which a lift mechanism is utilized to contact the shaped mating surface of the lower heating element to bring it in contact with the bottom surface of the wellplate.

### DETAILED DESCRIPTION

It is to be appreciated that embodiments of the systems and methods discussed herein are not limited in application to the details of construction and the arrangement of components set forth in the following description or illustrated in the accompanying drawings. The methods and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiments.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present systems and methods or their components to any one positional or spatial orientation.

Embodiments of the present disclosure are directed to a method of performing an incubation process in which a sample, such as a mixture of a biological sample containing analytes to be measured combined with reagents, chemicals, or other solutions, is caused to heat by being subjected to a first, higher temperature for a relatively short period of time and then maintained at a second, lower temperature for a relatively longer period of time. Further embodiments are directed to an incubator that is able to perform such a two-stage incubation method for processing samples. In one embodiment, the incubator includes a chamber and a movable heating block that is designed to conductively engage a wellplate carrier containing samples. In another embodiment, the incubator includes two chambers and an internal method of moving the sample between the two chambers. In yet another embodiment, the incubator includes two chambers and a method of removing the sample from one chamber and placing it into the other chamber.

In traditional passive convective air incubation and forced air incubation, heating of a diagnostic sample from room temperature to 37° C. is done slowly and with small thermal gradients across the sample. Thus, despite the slowness of the process the user benefits from a lack of possibility of overshooting the desired temperature and a simplicity of design. Water bath incubation shares these benefits. The present disclosure does not rely predominantly on convection of the external fluid (air or water), but rather relies on conduction through very thin air gaps and multiple-temperature incubation through very carefully controlled temperatures and times to achieve very fast heating without overheating any portion of the sample.

In the present disclosure, carefully designed surfaces are mated together forming very narrow air gaps. While in an idealized form, no air gaps would exist, thermal expansion and contraction requires that gaps exist. Further, imperfections in machining and molding further ensure that small air gaps will exist between the sample holder and the mating heated surface. Careful machining and molding and good mechanical design enables air gaps which are small. When heating the sample holder with a mating heated surface, the air in these very narrow air gaps experiences minimal convection and heating is primarily conductive through this thin layer of air. Suitable designs further trap the air and help to further suppress convection out of the gap. While intimate, perfect contact is not possible without applying high pressures to press the sample holder to conform to the shape of the mating heated surface, the two surfaces essentially contact in that lower surfaces of the sample holder have at most a very thin air gap.

The present disclosure further employs two-stage heating. In the first stage, the sample experiences rapid heating through contacting with a first, higher temperature. In one embodiment, this first higher temperature is 70° C. This first stage heats the sample rapidly and somewhat non-uniformly. Thus, the temperature, duration, shaped surface, and sample holder are suitably selected to ensure adequate heating without overheating of any portion of the fluid. The present disclosure teaches specific values which are suitable for common well-plate disposables as well as custom well-strip disposables. The present disclosure further presents the methodology for selecting parameters for alternate disposables.

The second stage of heating is a simple incubation at or near the target temperature. In this stage, temperature gradients in the sample gradually reduce through conduction as well as through convection of the sample. Further, the average temperature of the sample further continues to approach the target temperature. While timing in the first stage is fairly critical, because heat transfer occurs quickly, timing in the second stage is much less critical as the sample slowly approaches the target temperature. This allows for sufficient incubation time as well as robustness against process variations that often occur when automating complex assays.

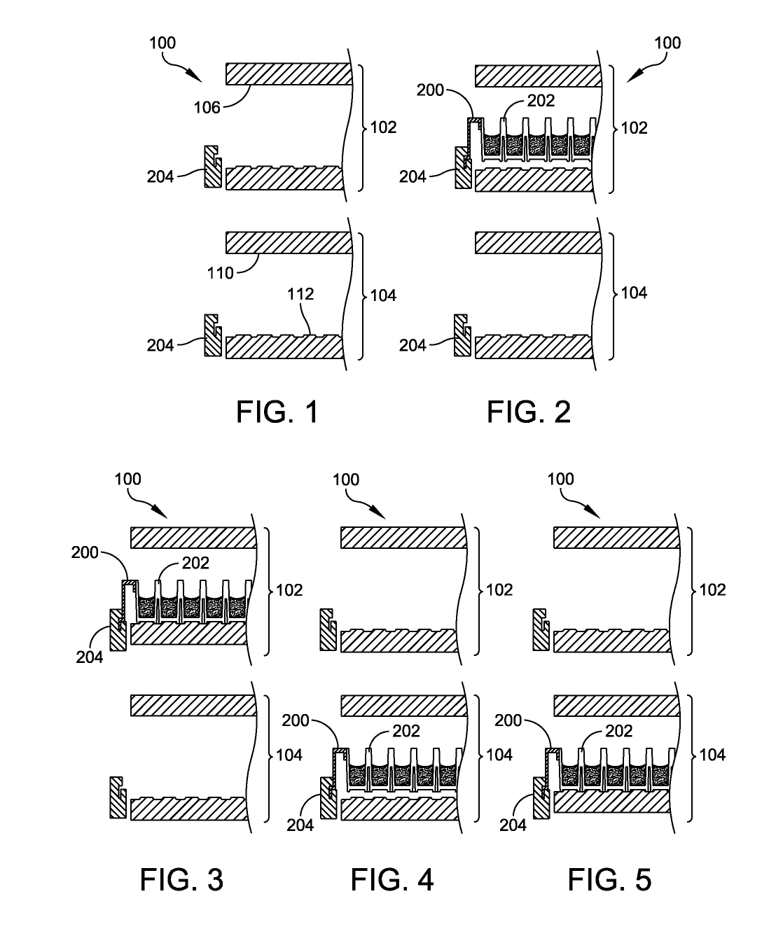

Turning now to the drawings, and more particularly to FIGS. 1-5, a method of incubating a sample is shown. FIG. 1 illustrates a portion of an incubator generally indicated at 100 in cross section having a top chamber 102 and a bottom chamber 104. In one embodiment, the top incubator chamber 102 may be operated at a higher temperature, e.g., 70° C. The lower incubator chamber 104 is maintained at an incubation temperature of 37° C. The top incubator chamber 102 has an upper heated surface 106 and a lower heated surface 108. Similarly, the bottom chamber 104 had an upper heated surface 110 and a lower heated surface 112.

FIG. 2 illustrates the top incubator chamber 102 having a holder or carrier 200 that is configured to receive a microplate 202, which may be referred to herein as a wellplate. The wellplate 202 is designed to receive a plurality of samples, such as in-vitro diagnostic testing and screening samples, requiring incubation. As shown, the wellplate 202 to be incubated is first received within the wellplate carrier 200 of the top incubator 102, which is disposed between the upper heated surface 106 and the lower heated surface 108. As shown, each of the top incubator chamber 102 and the bottom incubator chamber 104 includes a frame 204, which is configured to support the wellplate 202.

FIG. 3 illustrates the lower heated surface 108 of the top incubator chamber 102 being lifted so that the lower heated surface essentially contacts the bottom surface of the wellplate 202. As shown, the lower heated surface 108 is maintained at a temperature of 70° C. to quickly heat the samples contained within the wellplate 202. Direct conductive heating is the dominant heat transfer mechanism.

FIG. 4 illustrates the movement of the wellplate 202 after incubating in the top incubator chamber 102 for a relatively short period of time, e.g., 40-45 seconds, from the top incubator chamber to the bottom incubator chamber 104. In one embodiment, the bottom incubator chamber 104 may be operated a lower temperature, e.g., 37° C., and the wellplate 202 is immediately placed into the lower incubator chamber.

FIG. 5 illustrates the lower heated surface 112 of the lower incubator chamber 104 being lifted so that the lower heated surface essentially contacts the bottom surface of the wellplate 202. In one embodiment, the wellplate 202 remains in the lower incubator chamber 104 for a remaining time of the incubation period, which may be approximately 4.25 minutes.

The method of embodiments of the present disclosure is a two-step or multi-step incubation process, with a final step incubating at a target temperature and previous step(s) operating at elevated temperature(s).

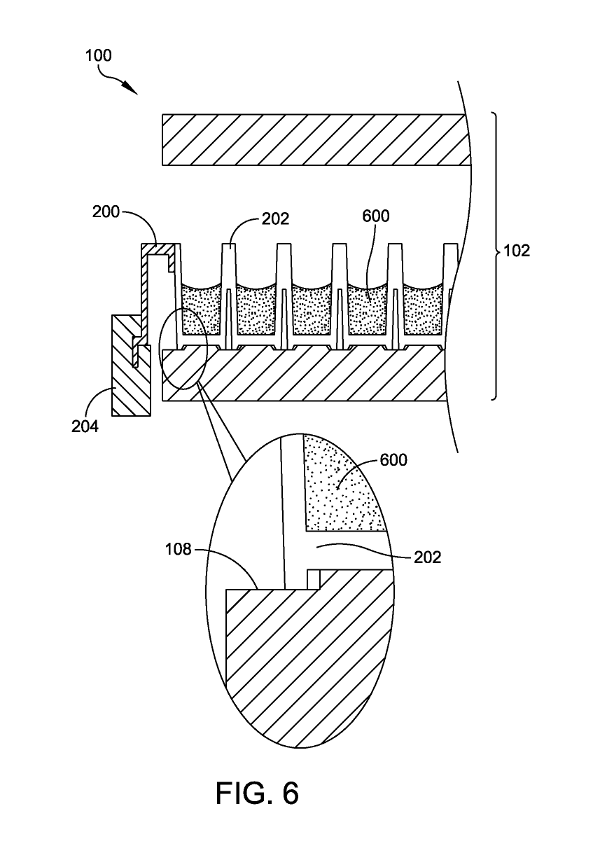

FIG. 6 illustrates a lower heated surface, such as the lower heated surface 108 of the top incubator chamber 102, having top geometrical features that are designed to mate with a lower surface of the wellplate 202 to achieve the desired heating rate and uniformity within the fluid of the wells. For example, FIG. 6 illustrates the bottom surface of the wellplate 202 having a plurality of indentations that correspond to and mate with a plurality of projections formed in a top surface of the lower heated surface 108. The wellplate 202 is configured to support a plurality of samples, each indicated at 300.

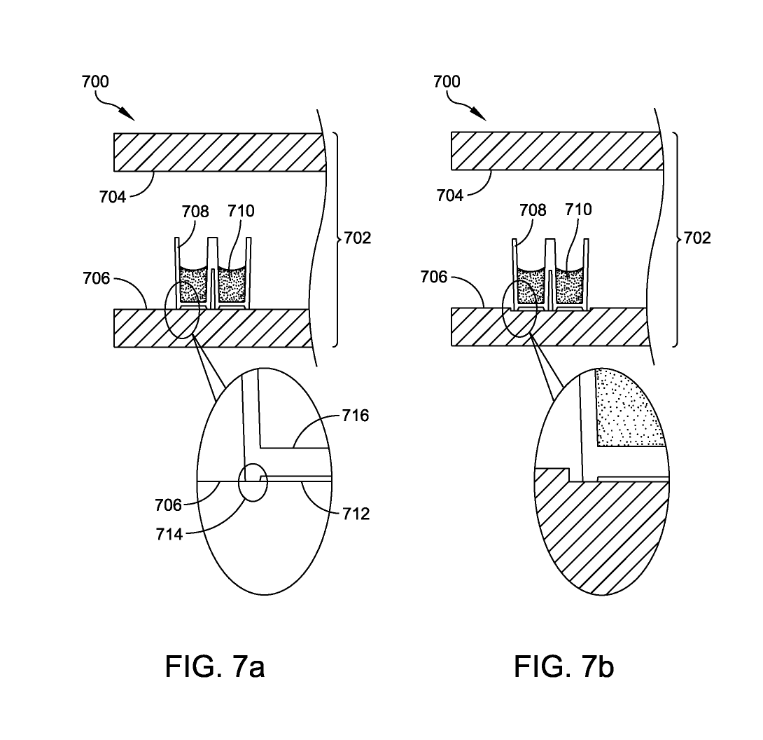

FIGS. 7a and 7b illustrate an incubator generally indicated at 700 having a chamber 702 with an upper heated surface 704 and a smooth lower heated surface 706, and a custom well-strip 708 (or wellplate) designed receive samples 710 therein and to achieve suitable thermal performance without movement or shaping of the lower heated surface. It should be noted that imaging of wells of the well-strip 708 generally requires that a lower surface of the well-strip have ridges that prevent scratching of the outside bottom of the wells. The resulting air gaps 712 can reduce performance unless the well-strip 708 is designed with this application in mind. Further, shaping of the lower heating surface 706 may result in a requirement that a lower heated surface have a lifting mechanism. To eliminate the need for a lifting mechanism while achieving high performance, a smooth lower heated surface may be employed with a custom well-strip with several characteristics: (a) the outer lower surface of each well is flat and optically clear; (b) at one or multiple areas a “nub” 714 extends down to prevent scratching of the lower surface; (c) the “nub” is very short in height, only slightly larger than system tolerances; and (d) the inner lower surface of each well is flat and optically clear.

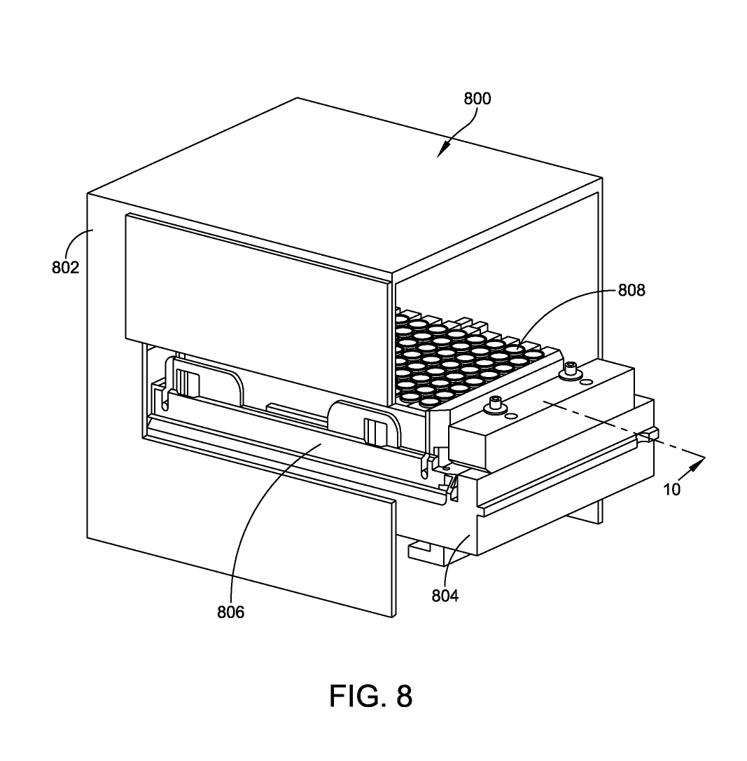

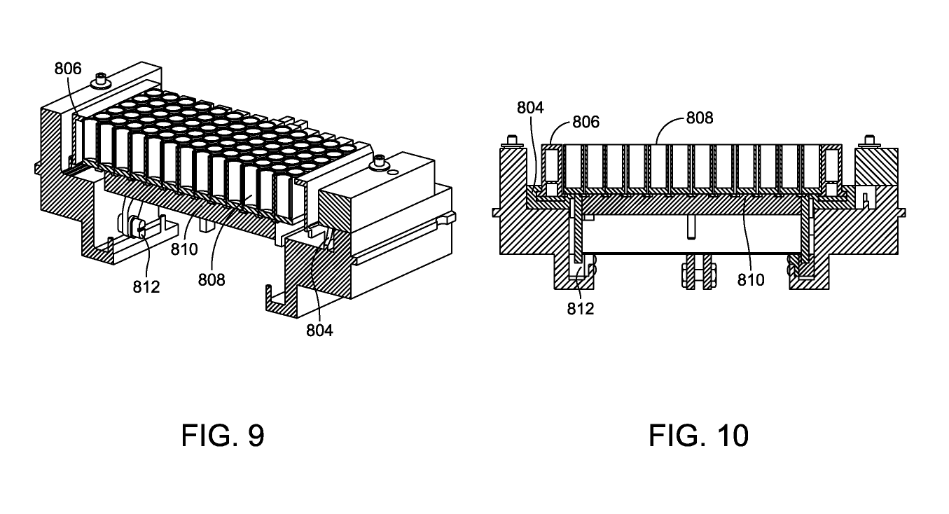

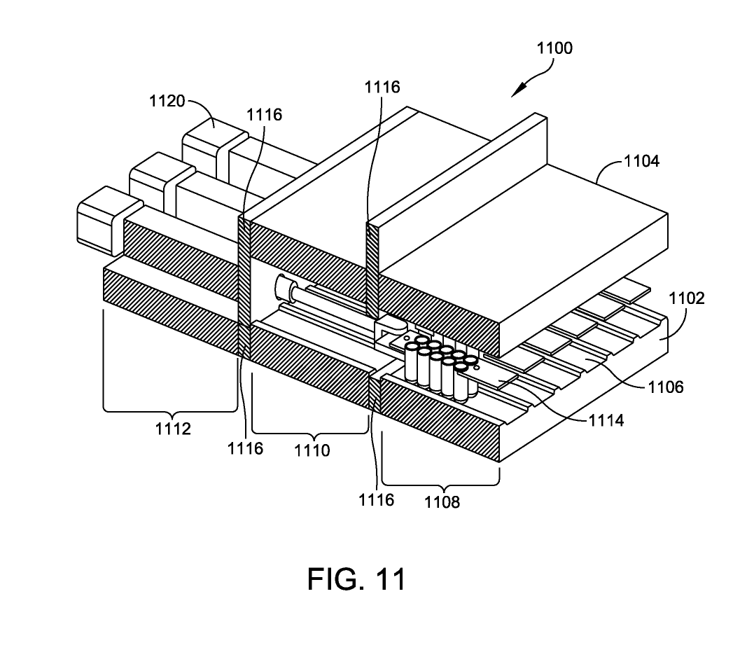

FIGS. 8-10 illustrate the working components of an incubator generally indicated at 800 of an embodiment of the present disclosure. A container 802 of the incubator 800 is partially shown in FIG. 8 so that the working components may be easily viewed. The container 802 may be constructed in any well-known manner. As shown, the incubator 800 includes a frame 804 for receiving a wellplate carrier 806, and a wellplate 808 supported within a wellplate carrier. The wellplate 808 is configured to receive a plurality of samples requiring incubation. The incubator further includes a lower heated surface 810 that is movable in a vertical direction from a position in which the lower heated surface is spaced sufficiently from a bottom surface of the wellplate carrier 806 to allow for insertion and removal of the wellplate 808 and carrier and a position in which the lower heated surface essentially contacts the bottom surface of the wellplate. The arrangement is such that when the lower heated surface 810 essentially contacts the wellplate, the lower heated surface heats the wellplate 808 through conductive heat transfer through direct contact or through a very thin layer of air. An upper heated surface, although not shown, is further provided to heat the wellplate 808 containing the samples.

A lower heated surface lifting/lowering mechanism 812 is further provided to move the lower heated surface 810, thus enabling lateral movement of the wellplate 808 and carrier 806 without interference from the lower heated surface. In one embodiment, the lower heated surface lifting/lowering mechanism 812 includes a drive motor having an eccentric linkage or coupling that is configured to move the lower heated surface 810. It should be understood that any suitable mechanism may be provided to move the lower heated surface 810. When operated to lift the lower heated surface 810, the lower heated surface essentially contacts the wellplate 808 to conductively transfer heat to the wellplate. When disengaged, the lower heated surface 810 still transfers heat but at a lower rate since the heat is being primarily transferred by convection and radiation.

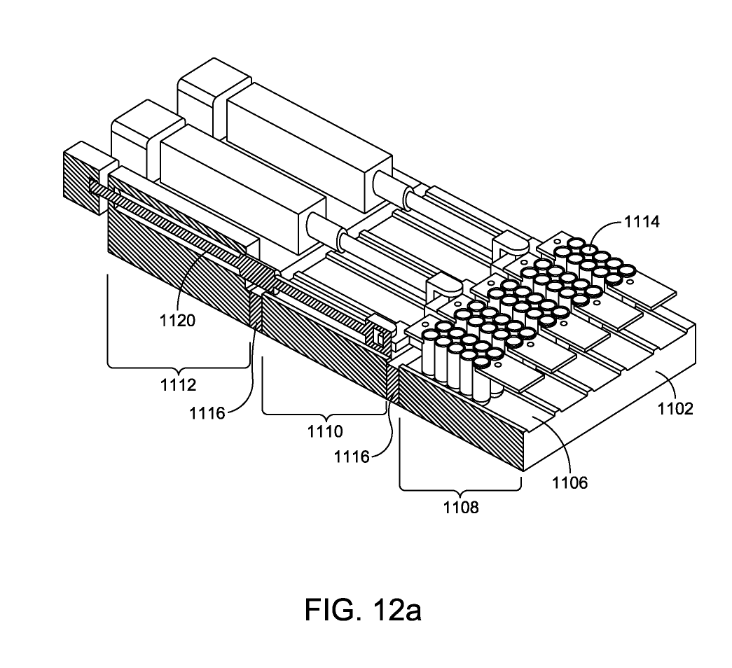

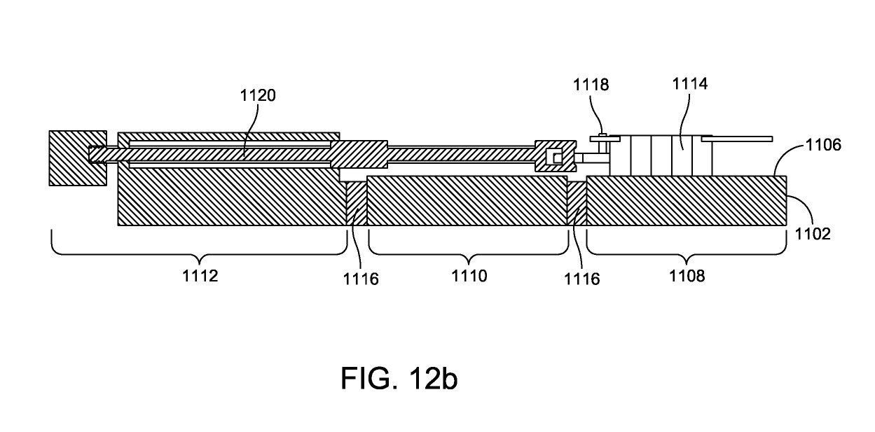

FIGS. 11, 12a and 12b illustrate a system generally indicated at 1100 comprising a custom well-strip sample holder 1102 and an incubator 1104 that is designed to quickly heat the sample holder to approximately 37° C. and to hold the temperature at 37° C. The custom well-strip sample holder 1102 has flat inner and outer lower surfaces 1106 for imaging and a very short nub or lip at the outer well bottom to create a very thin air gap and prevent scratching of the lower surface. As shown, the system 1100 has a front chamber 1108, a middle chamber 1110 and a rear chamber 1112. The front chamber 1108 of the incubator 1104 has a door (not shown) which opens to accept well-strips 1114 and closes after the well-strips are inserted. The front chamber 1108 of the incubator 1104 is held at 37° C. The well-strips 1114 are deposited into recessed tracks in the lower heated surface 1106 of the front chamber 1108. A moveable thermal insulating barrier 1116 separates the front chamber 1108 from a rear incubation chamber 1112 which is held at 70° C. When the well-strips 1114 are placed in the front incubation chamber 1108, each well-strip is captured by a pin 1118 on a driving mechanism 1120 which is able to slide the well-strip in the track, between the front incubation chamber and the rear incubation chamber 1112. After the well-strip 1114 is placed in the front incubation chamber 1108, the barrier 1116 is moved, the well-strip is moved into the rear incubation chamber 1112, and the barrier is replaced into its standard position. After the well-strip 1114 is incubated for the prescribed period of time in the rear incubation chamber 1112, such as 40 seconds, the barrier 1116 is again moved, the well-strip is slid into the front incubation chamber 1108, and the barrier is replaced. The incubator 1104 has multiple tracks enabling it to process multiple well-strips 1114. Employing separate driving mechanisms 1120, the well-strips 1114 can be processed asynchronously. By employing internal dedicated mechanisms for movement of the well-strips 1114 between the two incubation chambers, the process does not require external resources, such as an external transport mechanism, to act at highly controlled times. Thus, this incubator 1104 will easily integrate into a complex in-vitro diagnostic instrument with many modules and constraints on the use of those resources.

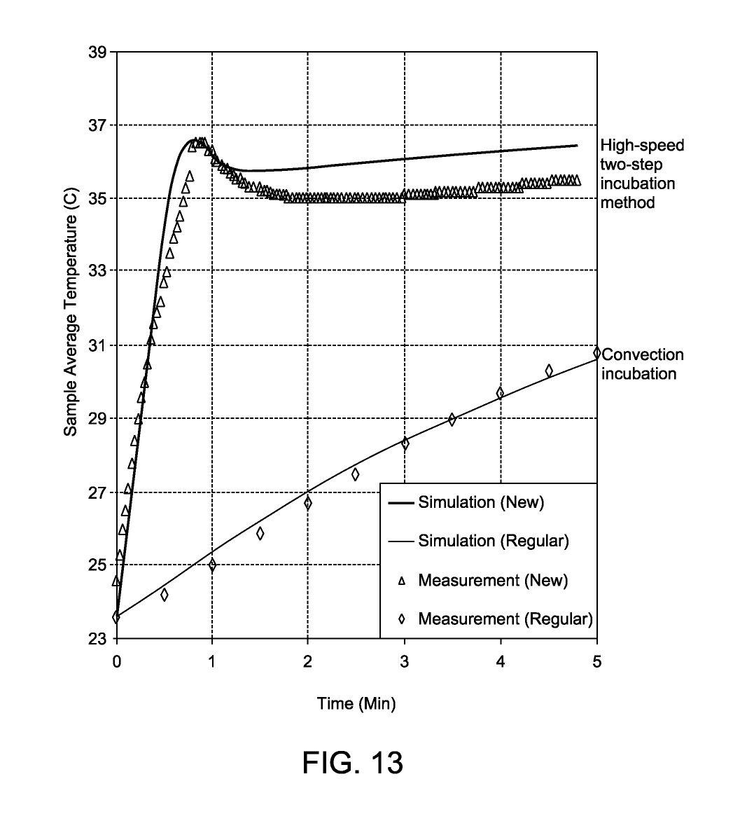

FIG. 13 illustrates a graphical representation of performing the method of embodiments of the present disclosure. As shown, the sample is heated at an elevated temperature, e.g., 70° C., for less than a minute, e.g., 45 seconds, during a first phase of heating. With reference to FIGS. 1-5, the sample may be positioned within a wellplate placed in the top incubator chamber. After the first phase, the sample is transferred to a bottom incubator chamber held at or near the target temperature, e.g., 37° C., for a length of time, e.g., 4.25 minutes, to maintain the sample at the desired temperature. FIG. 13 further illustrates a known convection heating method in which the sample is heated in a single step in a simple convection oven incubator held at single temperature, e.g. 37° C., without essential contact between the shaped lower surface of the sample holder and a shaped upper surface of a lower heated surface. With such a known method, the duration time is far greater to achieve the desired incubation temperature, e.g., 37° C.

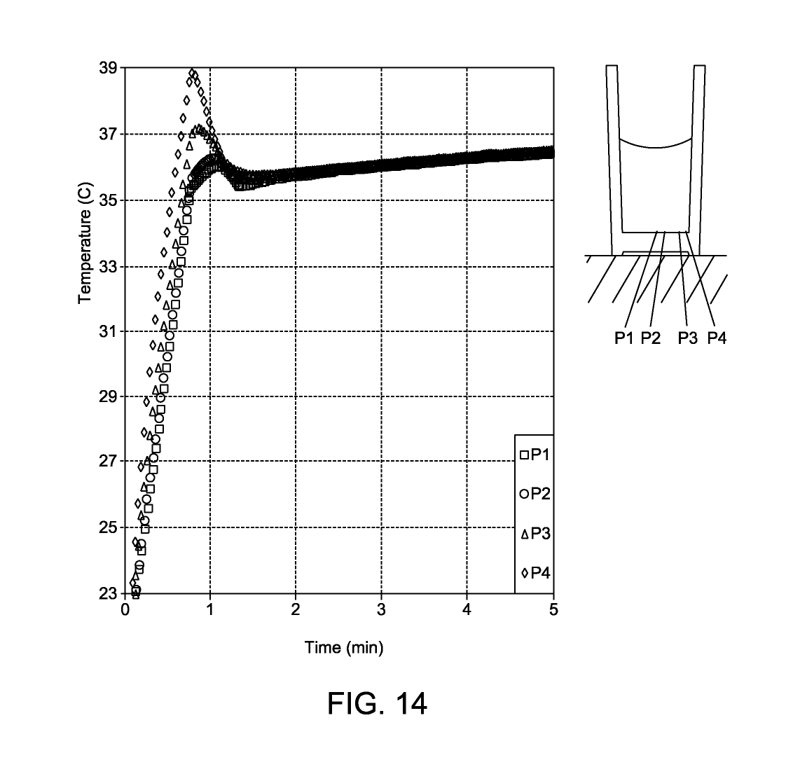

FIG. 14 is a graph showing the temperature-time series for fluid at several points near the lower surface of a flat-bottom well in the incubator of FIG. 6.

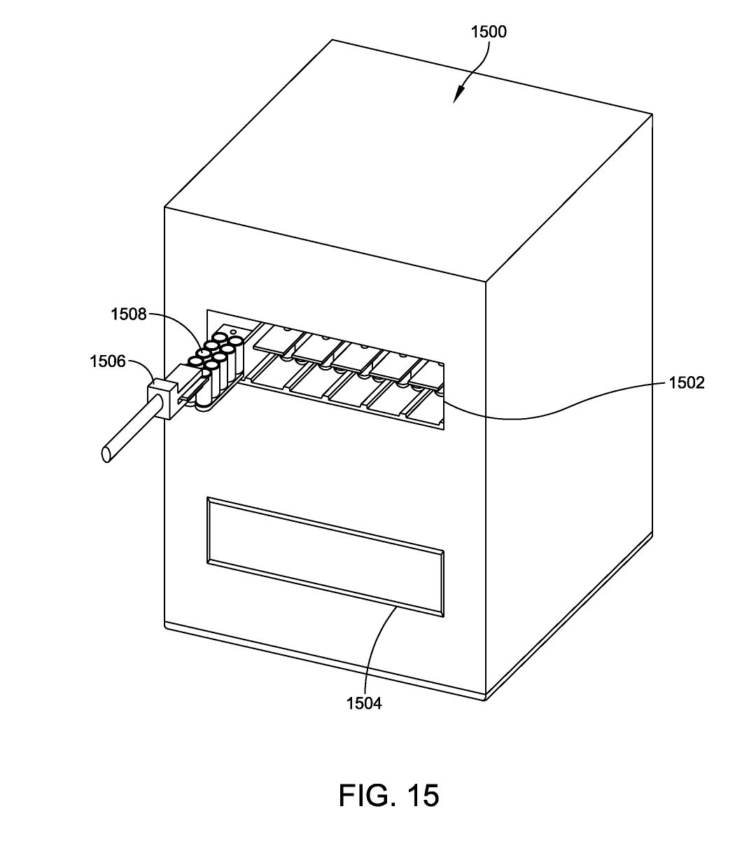

FIG. 15 is view of an exemplary embodiment of an incubator generally indicated at 1500 having a top chamber 1502 and a bottom chamber 1504. The incubator 1500 utilizes an external transport mechanism 1506 to transport a custom well-strip 1508 between two chambers of the incubator. The custom well-strip 1508 has flat inner and outer lower surfaces for imaging and a very short nub or lip at the outer well bottom to create a very thin air gap and prevent scratching of the lower surface. The incubator 1500 comprises two incubation chambers in which the top chamber 1502 is held at 70° C. and the lower chamber 1504 is held at or near 37° C. The external transport mechanism 1506 first places the well-strip 1508 into the top chamber 1502 for a precisely controlled period of time, e.g. 45 seconds, where the sample is more rapidly heated in a precisely controlled manner so as to reach a narrow window of the target temperature without overheating any portion of the sample. The external transport mechanism 1506 then transfers the well-strip sample holder 1508 from the top chamber 1502 to the lower chamber 1504 where the sample approaches equilibrium and continues its incubation at or near the target temperature, e.g. 37° C.

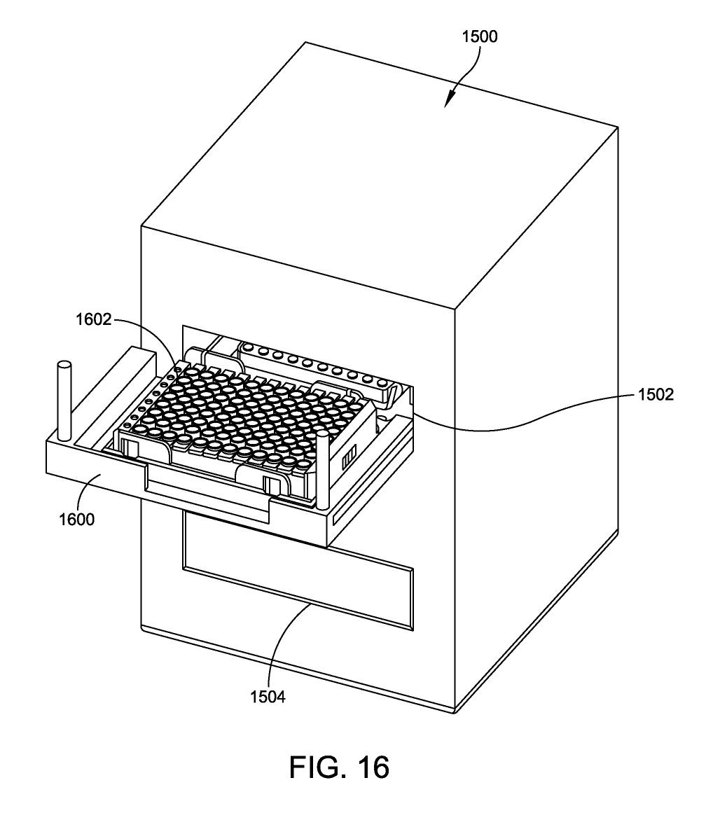

FIG. 16 is view of an exemplary embodiment which utilizes an external transport mechanism 1600 to transport a wellplate 1602 between two chambers 1502, 1504 of the incubator 1500. The wellplate 1602 is held within the carrier which extends below the lower surface of the wells, requiring the use of a moveable lower heated surface. The incubator 1500 comprises two incubation chambers in which the top chamber 1502 is held at 70° C. and the lower chamber 1504 is held at or near 37° C. The external transport mechanism 1600 first places the wellplate sample holder 1602 into the top chamber 1502 for a precisely controlled period of time, e.g. 45 seconds, where the sample is more rapidly heated in a precisely controlled manner so as to reach a narrow window of the target temperature without overheating any portion of the sample. The external transport then transfers the wellplate sample holder 1602 from the top chamber 1502 to the lower chamber 1504 where the sample approaches equilibrium and continues its incubation at or near the target temperature, e.g. 37° C.

It should be understood that the method of the present disclosure may be achieved by an incubator having a single chamber that is capable of heating the sample with conductive heat transfer at a first, higher temperature (e.g., 70° C.) for a short period of time (e.g., 45 seconds), and incubating the sample at a second, lower temperature (e.g., 37° C.) for a longer period of time (e.g., four or so minutes).

Having thus described several aspects of at least one embodiment of this disclosure, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

1. An incubator comprising:
a container having an interior chamber capable of receiving a sample holder having samples;
a lower heated surface disposed within the interior chamber of the container; and
a movement mechanism coupled to the container and the lower heated surface, the movement mechanism being configured to move the lower heated surface to a position in which the lower heated surface essentially contacts the sample holder.

2. The incubator of claim 1, wherein the movement mechanism includes a motor configured to move the lower heated surface between an engaged position in which the lower heated surface engages the sample holder and a disengaged position in which the lower heated surface is spaced from the sample holder.

3. The incubator of claim 2, wherein the motor includes an eccentric coupling secured to the lower heated surface.

4. The incubator of claim 1, wherein the lower heated surface is positioned below the sample holder.

5. The incubator of claim 4, further comprising an upper heated surface positioned above the sample holder.

6. The incubator of claim 1, wherein the sample holder includes a bottom surface configured to mate with the lower heated surface.

7. The incubator of claim 1, wherein the interior chamber includes a top chamber having the sample holder, the lower heated surface and the movement mechanism, and a bottom chamber.

8. A method of rapidly heating and incubating a sample for biochemical or immunological testing, the method comprising:
heating the sample with conductive heat transfer at a first, higher temperature for a short period of time; and
incubating the sample at a second, lower temperature for a longer period of time.

9. The method of claim 8, wherein the conductive heat transfer is achieved by bringing a lower heated surface into close proximity or contact to a sample holder configured to hold one or more sample, wherein the lower heated surface and/or the sample holder are designed to achieve essential contact with each other.

10. The method of claim 9, wherein the heating of the sample with conductive heat is achieved by an incubator comprising a container having an interior chamber, the interior chamber being configured to receive the sample holder having samples, a lower heated surface disposed within the interior chamber of the container, and a movement mechanism coupled to the container and the lower heated surface, the movement mechanism being configured to move the lower heated surface to a position in which the lower heated surface essentially contacts the sample holder.

11. The method of claim 9, wherein the interior chamber includes a top chamber having the lower heated surface and the movement mechanism, and a bottom chamber, and wherein heating the sample at the first temperature is achieved within one chamber and heating the sample at the second temperature is achieved within the other chamber.

12. The method of claim 8, wherein the first temperature is greater than 50° C. and less than 90° C.

13. The method of claim 12, wherein the first temperature is applied for less than one minute.

14. The method of claim 12, wherein the first temperature is applied for approximately 45 seconds.

15. The method of claim 12, wherein the second temperature is between 34° C. and 43° C.

16. An incubator comprising:
a first chamber held at a first temperature, capable of accepting a sample holder containing a sample;
a second chamber, held at a second, higher, temperature, capable of accepting a sample holder containing a sample; and
a heated surface, within the second chamber, capable of achieving essential contact with a sample holder containing a sample.

17. The incubator of claim 16, further comprising a mechanism for moving the sample holder between the first chamber and the second chamber.

18. The incubator of claim 16, wherein the first chamber is held between 34° C. and 43° C.

19. The incubator of claim 16, wherein the second chamber is held between 50° C. and 90° C.

20.-24. (canceled)

25. The incubator of claim 16, wherein a heated surface within the second chamber is within 0.25 mm of a flat lower bottom surface of the sample holder for at least 20 seconds.

26. An incubation system comprising:
a transparent sample holder, with a flat lower surface, which is able to achieve essential contact with a flat heating surface without scratching the flat lower surface; and
an incubator comprising:
a first chamber held at a first temperature, capable of accepting the transparent sample holder containing a sample,
a second chamber, held at a second, higher, temperature, capable of accepting the transparent sample holder containing a sample, and
a heated surface, within the second chamber, capable of achieving essential contact with a sample holder containing a sample.

27. The incubator of claim 26, wherein the first chamber is held between 34° C. and 43° C.

28. The incubator of claim 26, wherein the second chamber is held between 50° C. and 90° C.
